(19) United States
(12) Patent Application Publication
(10) Pub. No.: US 2007/0109814 A1
(43) Pub. Date: May 17, 2007

(54) Fluorescent light air freshener

(76) Inventor: Michael A. Logan, Oviedo, FL (US)

Correspondence Address: William M. Hobby, III;157 E. New England Avenue, #375, Winter Park, FL, 32789, US

(21) Appl. No.: 11/256,143

(22) Filed: Oct. 24, 2005

(51) Int. Cl. A61M 16/00

(52) U.S. Cl. 362/643; 392/392

(57) ABSTRACT

A fluorescent light air freshener dispenser has a fluorescent lamp having a coiled fluorescent tube attached to an electronic ballast. A permeable container is sized to fit in the coiled fluorescent tube and has a reservoir for holding an evaporative scent composition therein. A container supporting clip is attached to the permeable container and supports the permeable container in the center of the coiled fluorescent tube so that a fluorescent lamp dispenses the evaporative air freshener when the fluorescent lamp is activated.

### BACKGROUND OF THE INVENTION

The present invention relates to room air fresheners and especially to room air fresheners that are placed in and attached to a coiled fluorescent tube of a fluorescent light.

In the past, there have been a wide variety of room air fresheners and most of these typically use some type of heater for heating an evaporative air freshener composition or utilize some existing air flow, such as produced by a small fan or the like. One source for obtaining heat for a room air freshener can be from an incandescent lightbulb which operates by the passage of an electric current through a filament to produce a white hot filament producing the light for the lightbulb. Fluorescent bulbs, on the other hand, typically do not produce any significant amount of heat in the tube itself. A fluorescent lamp is a gas discharge tube in which the inner surface of the wall of the tube is coated with a light emitting or fluorescent substance. The tube is filled with a low pressure gas. Electrodes are placed at each end of the fluorescent tube. The cathode electrode is heated and ejects electrons therefrom through the tube towards the anode electrode at the other end of the tube. The collision of the electrons with atoms of the gas produce radiation, such as ultraviolet rays, which in turn strike the fluorescent coating on the walls of the tube to produce a visible light. Most of the heat generated by a fluorescent lamp is produced in the ballast by the heating of the cathode for producing the electrons in the tube. The amount of heat produced is generally substantially less than that of an incandescent bulb for the same amount of generated visible light. It is therefore not been considered a useful heating element.

One example of a room air freshener using air currents passing over the air freshener composition can be seen in my prior U.S. Pat. No. 6,241,219 dated Jun. 5, 2001 for an Air Circulator Room Deodorizer. In this patent, an air circulator room deodorizer has a permeable container that is positioned with an air circulator attachment in the air flow from a room circulator, such as a room fan or vent, and has a deodorant pack that releases a fragrance selectively into the air flow. A permeable container is attached to the chain of a ceiling fan or the like so that the air flow from the ceiling fan passing over the permeable container evaporates the air freshener composition.

In the present application, a fluorescent light air freshener dispenser is provided for a coiled fluorescent tube of the type that is typically made with a ballast in a base and which is threadedly attached to a typical incandescent lamp having a threaded socket for attaching an incandescent light bulb. The placement of the air freshener dispenser directly inside the coiled fluorescent lamp advantageously places the permeable container air freshener directly over the ballast and directly over the electrodes for the fluorescent tube. Thus, the air freshener dispenser is operative whenever the fluorescent lamp is switched on and is advantageously concealed from view. This avoids the unsightly presence of an air freshener being plugged into an electrical outlet and which might contain a small heater to heat an evaporative composition.

### SUMMARY OF THE INVENTION

A fluorescent light air freshener dispenser has a fluorescent lamp having a coiled or looped fluorescent tube attached to an electronic ballast. A permeable container is sized to fit in the fluorescent tube and has a reservoir for holding an evaporative scent composition therein. A container supporting clip is attached to the permeable container and supports the permeable container in the center of the coiled or looped fluorescent tube so that a fluorescent lamp dispenses the evaporative air freshener when the fluorescent lamp is activated. The evaporative permeable container can have a wick extending from the reservoir for dispensing a liquid scent composition while the fluorescent light electronic ballast has a threaded base having an electrical contact on the end thereof for threadedly attaching to a lamp stand. The container support clip can be removably attached to the permeable container with a removable lid covering the reservoir. The removable lid can have a plurality of openings therein for releasing the evaporative scent from the permeable container. The container supporting clip can have an opening in the center thereof for attaching the lid through and can have a plurality of clips for removably attaching the supporting clip to a coiled or plurality of looped fluorescent tubes.

### BRIEF DESCRIPTION OF THE DRAWINGS

Other objects, features, and advantages of the present invention will be apparent from the written description and the drawings in which:

FIG. 1 is a side elevation of a fluorescent light air freshener dispenser attached to a lamp;

FIG. 2 is a side elevation of a fluorescent light air freshener dispenser mounted in a coiled fluorescent tube;

FIG. 3 is an exploded view of the fluorescent light air freshener dispenser of FIG. 2; and

FIG. 4 is an exploded view of a second embodiment of a fluorescent light air freshener dispenser.

### DESCRIPTION OF THE PREFERRED EMBODIMENT

Referring to the drawings, FIGS. 1, 2 and 3, a fluorescent light air freshener dispenser 10 is illustrated attached to a coiled fluorescent tube 11 extending from an electronic ballast base 12. The ballast 12 can be seen as having a threaded base 13 with a center contact 14 similar to that found on incandescent light bulbs. The fluorescent light is designed for attachment to a lamp 15 having an incandescent lamp socket 16 with internal threads to which the threaded base 13 of the fluorescent tube ballast 12 can be threaded. The lamp 15 also has a lamp shade support 17 attached to the lamp stand 15. The coiled fluorescent tube 11 can be seen having two ends 18 and 20, each of which will have an electrode mounted at the end thereof. A permeable container 10 can be shown as having a base 21 and a permeable lid 22. The base 21 can be transparent and can have a reservoir 23 therein which may hold a liquid or solid or any other evaporative scent or air freshener.

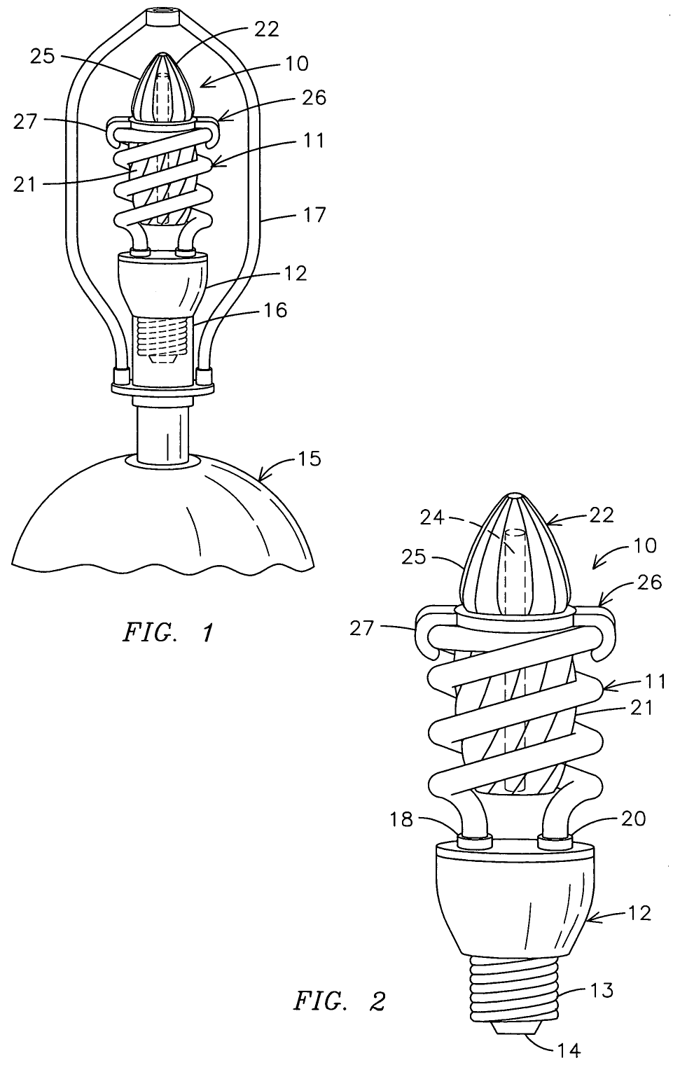

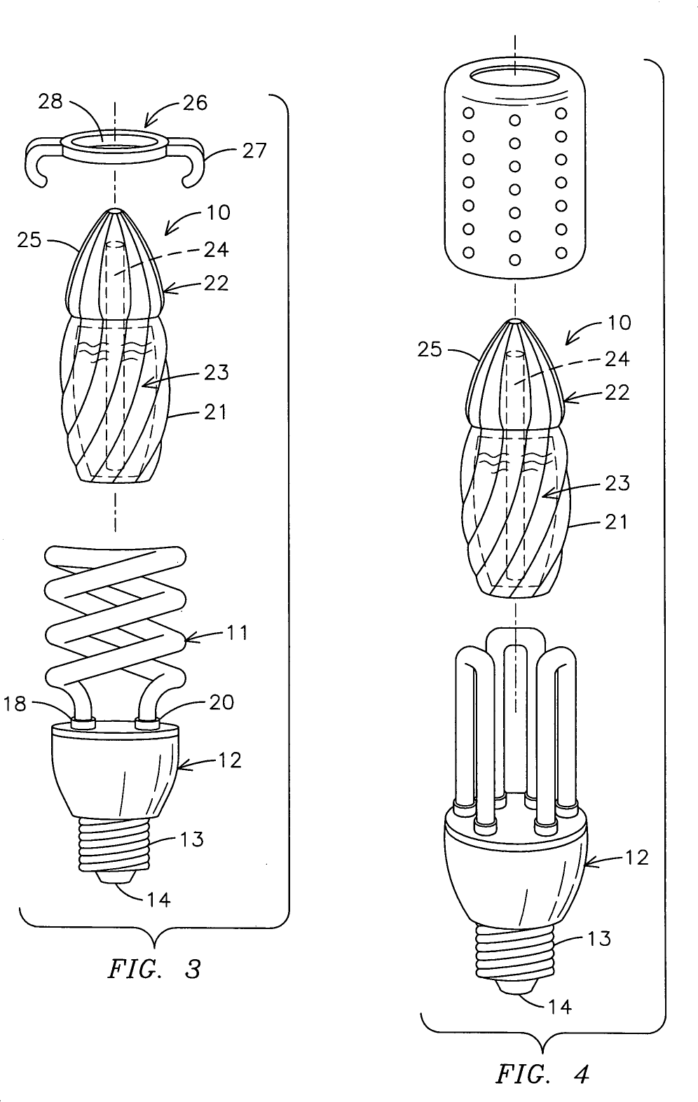

As illustrated in FIG. 3, the reservoir 23 has a wick 24 extending therefrom up into the lid 22. The lid 22 has a plurality of open spaces 25 spaced around the lid to allow the evaporation of the air freshener composition into the surrounding air. A polymer container supporting clip 26 has a pair of fluorescent tube clips 27 for clipping onto the coiled fluorescent tube 11. An open center 28 is used in attaching the tube clip to the permeable container 10. The attachment can be by the removal of the lid 22 and then threading the lid 22 through the opening 28 to the container base 21, as shown in FIGS. 1 and 2.

In operation, the permeable container 10 reservoir 23 can be filled with an air freshener or scent composition and with the wick 24 extending therefrom. The container supporting clip 26 can then be placed on the top of the container base 21 and the permeable lid 24 threaded thereto to clamp the container supporting clip 26 to the permeable container 10. The permeable container 10 can then be placed in the center of the coiled tube 11 and the clip 27 clipped onto the tube 11, as shown in FIGS. 1 and 2. Thus, when the lamp 15 is activated or switched on, the fluorescent tube 11 is lighted. The ballast 12 will produce a small amount of heat directly beneath the container 21 while the tube 11 will produce a small amount of radiation directly into the transparent container 10. This will slightly heat the evaporative composition in the reservoir of the container 10 which may or may not have the wick 24 therein. The composition can evaporate through the openings 25 of the lid 22. Other types of permeable containers can also be utilized without departing from the spirit and scope of the invention. For instance, the permeable container 10 can be of a solid material that is impregnated with the air freshener or scent composition for evaporation therefrom.

Turning now to FIG. 4, an alternate embodiment of the fluorescent light air freshener dispenser is illustrated having an air freshener dispenser 10 which may be a permeable container having the base 21 and a permeable lid 22. The base 21 can be transparent and can have a reservoir 23 therein which may hold a liquid or solid or other evaporative scent or air freshener. The lid may also have a plurality of open spaces 25 therein and may contain a wick 24 as desired. A container 10 is shaped to fit within a plurality of looped or generally U-shaped fluorescent tubes 30 which are attached to a fluorescent tube electronic ballast 12 having a threaded base 13 and a center electric contact 14. This type of fluorescent lamp may come with a translucent cover 31 which, in the present embodiment may have a plurality of apertures 32 in the side thereof and may have an open top 33 to allow the escape of the air scent. The fluorescent lamp of this embodiment also comes without any type of cover, such as 31, in which case a container 10 is inserted between the looped fluorescent tubes 30. The container 10 may be held in place with a clip 34 which can be shaped with a flange to rest on top of the looped fluorescent tubes 30 or to clip thereonto as desired.

It should be clear at this time that a fluorescent light air freshener dispenser has been provided which advantageously can be attached to a coiled fluorescent tube of a fluorescent light of the type for attaching to a standard incandescent lamp fixture. However, the present invention should not be construed as limited to the forms shown which are to be considered illustrative rather than restrictive.

I claim:

1. A fluorescent light air freshener dispenser comprising:
a fluorescent lamp having a fluorescent tube attached to an electronic ballast having a threaded base having an electrical contact thereon for threadedly attaching to a lamp shade;
a permeable container sized to fit onto said fluorescent tube, said permeable container having a reservoir for holding an evaporative scent composition therein; and
a container supporting clip attached to said permeable container and supporting said permeable container on said fluorescent tube, whereby a fluorescent lamp dispenses said evaporative air freshener when said fluorescent light is activated.

2. The fluorescent light air freshener dispenser in accordance with claim 1 in which said permeable container has a wick extending from said reservoir for dispensing a liquid scent composition.

3. The fluorescent light air freshener dispenser in accordance with claim 2 in which said fluorescent tube is a coiled fluorescent tube.

4. The fluorescent light air freshener dispenser in accordance with claim 3 in which said container supporting clip is removably attached to said permeable container.

5. The fluorescent light air freshener dispenser in accordance with claim 4 in which said permeable container has a removable lid to allow access for filling said reservoir with a scent composition.

6. The fluorescent light air freshener dispenser in accordance with claim 5 in which said removable lid has a plurality of openings therein to allow for the evaporation of scent from said permeable container.

7. The fluorescent light air freshener dispenser in accordance with claim 6 in which said container supporting clip has an open center portion for attaching to said permeable container with said removable lid.

8. The fluorescent tube dispenser in accordance with claim 7 in which said container supporting clip has a plurality of clips for removably attaching said supporting clip to a coiled fluorescent tube.

9. The fluorescent tube dispenser in accordance with claim 8 in which said container supporting clip is a polymer clip.

10. A fluorescent light air freshener dispenser comprising:
a permeable container sized to fit in a coiled fluorescent tube, said permeable container having a reservoir and an opening thereinto for placing an evaporative scent composition therein; and
a container supporting clip attached to said permeable container and shaped for attachment to a coiled fluorescent tube to support said permeable container to said coiled fluorescent tube, whereby a fluorescent lamp can dispense an evaporative scent when said permeable container having an evaporative scent composition therein is attached to the fluorescent tube.

11. The fluorescent light air freshener dispenser in accordance with claim 10 in which said evaporative container has a wick extending from said reservoir for dispensing a liquid scent composition.

12. The fluorescent light air freshener dispenser in accordance with claim 11 in which said container supporting clip is removably attached to said permeable container.

13. The fluorescent tube dispenser in accordance with claim 12 in which said permeable container has a removable lid to allow access for filling said reservoir with a scent composition.

14. The fluorescent light air freshener dispenser in accordance with claim 13 in which said removable lid has a plurality of openings therein to allow for the evaporation of scent from said permeable container.

15. The fluorescent light air freshener dispenser in accordance with claim 14 in which said container supporting clip has an open center portion for attaching to said permeable container with said removable lid.

16. The fluorescent light air freshener dispenser in accordance with claim 15 in which said container supporting clip has a plurality of clips for removably attaching said supporting clip to a coiled fluorescent tube.

17. The fluorescent light air freshener dispenser in accordance with claim 16 in which said container supporting clip is a polymer clip.

18. The fluorescent light air freshener dispenser in accordance with claim 17 in which said permeable container is a polymer container having a removable lid.

19. The fluorescent light air freshener dispenser in accordance with claim 10 in which permeable container holds a solid evaporative scent.
